(19) United States
(12) Patent Application Publication
(10) Pub. No.: US 2022/0304831 A1
(43) Pub. Date: Sep. 29, 2022

(54) METHOD FOR CONTROLLING AN ARTIFICIAL KNEE JOINT

(71) Applicant: Otto Bock Healthcare Products GmbH, Wien (AT)

(72) Inventor: Dirk SEIFERT, Wien (AT)

(73) Assignee: Otto Bock Healthcare Products GmbH, Wien (AT)

(21) Appl. No.: 17/616,301

(22) PCT Filed: Jun. 4, 2020
(86) PCT No.: PCT/EP2020/065465

§ 371 Date: Dec. 3, 2021

(30) Foreign Application Priority Data
Jun. 5, 2019 (DE) 10 2019 115 098.1

(51) Int. Cl. A61F 2/64; A61F 2/70

(52) U.S. Cl. CPC A61F 2/64; A61F 2/70; A61F 2002/704; A61F 2002/7625

(57) ABSTRACT

The invention relates to a method for controlling an artificial knee joint which includes an upper part having an anterior side and a posterior side; a lower part mounted on the upper part so as to be pivotable about a knee axis and having an anterior side and a posterior side; a foot part arranged on the lower part; at least one sensor; a control device connected to the at least one sensor; and an actuator which is coupled to the control device and by means of which an achievable knee angle (KAmax) between the posterior side of the upper part and the posterior side of the lower part in the swing phase can be set by the control device.

The invention relates to a method for controlling an artificial knee joint comprising an upper part with an anterior face and a posterior face, a lower part which is mounted on the upper part so as to be pivotable about a knee axis and has an anterior face and a posterior face, a foot part arranged on the lower part, and an actuator via which an achievable knee angle between the posterior face of the upper part and the posterior face of the lower part at the end of a swing phase can be set.

Artificial knee joints are used in prostheses, orthoses and exoskeletons. An artificial knee joint has an upper part and a lower part, which are mounted so that they are pivotable relative to each other about a knee axis. In the simplest case, the knee joint is designed as a single-axis knee joint in which, for example, a bolt or two bearing points arranged on a pivot axis form an individual knee axis. There are also knee joints that do not form a fixed axis of rotation of the upper part relative to the lower part, but instead have either sliding or rolling surfaces or a large number of articulated arms that are connected to one another. So-called four-axis knee joints with spring devices and dampers have been described quite often in the prior art. There are also five-axis and six-axis knee joints. In the case of orthoses and exoskeletons, multi-axis designs of the artificial knee joints are the exception.

Prosthetic knee joints are often produced and supplied as a complete assembly with upper attachment means for securing a thigh stump or another device for securing the upper part on the patient, and fastening devices for securing a lower part, for example a lower leg tube or a prosthetic foot. In the case of orthoses and exoskeletons, fastening devices for securing the artificial knee joint to the patient can be arranged directly on an upper part and a lower part, for example in the form of straps, cuffs or shells, which are arranged on rails or external frame structures.

To influence the extension movement and/or flexion movement, it is known to arrange an actuator between the upper part and the lower part, for example in the form of a damper or a drive.

DE 10 2013 011 080 A1 relates to a method for controlling an orthopedic joint device of a lower extremity, with an upper part and a lower part articulated thereon, between which parts a conversion device is arranged via which, during a pivoting of the upper part relative to the lower part, mechanical work is converted from the relative movement and is stored in at least one energy store. The energy is fed back to the joint device with a time delay in order to support the relative movement, wherein the stored energy is converted back and the mechanical work is supplied in a controlled manner while the relative movement is supported. In addition to the conversion device, a separate damper in the form of a hydraulic or pneumatic damper can be provided, which is adjustable, such that the resistance during walking, both in the flexion direction and in the extension direction, can be influenced via the damper device.

U.S. Pat. No. 5,181,931 A relates to a pivot connection between two parts of an orthopedic device with an upper part and a lower part and with an adjustable mechanical extension stop.

EP 2 240 124 B1 relates to an orthopedic knee joint with an upper part on which upper attachment means are arranged, a lower part which is mounted pivotably on the upper part and has attachment means for orthopedic components, and a stop for limiting an extension movement. The stop is designed to be movable and is coupled to an adjustment device, which is in turn coupled to a control device that actuates the adjustment device, in accordance with sensor data, and changes the position of the stop in the sense that an extension stop is shifted forward for walking and drawn back for standing.

An artificial knee joint has a knee angle of 180° at the maximum extension allowed by the design; hyperextension, that is to say an angle on the posterior face of greater than 180°, is generally not provided. The rearward pivoting of the lower part with respect to the upper part is called knee flexion, while anterior pivoting is called extension. At the initial contact, the foot is set down on the ground at the end of the swing phase and the beginning of the stance phase. When walking on level ground, there is usually a heel strike, that is to say the foot is set down first with the heel. If the artificial knee joint remains in an extended, straight position, this leads to a direct transmission of force into the pelvis, which feels very unpleasant. Analogously to natural walking, a so-called stance phase flexion is therefore permitted or carried out in prostheses or orthoses, in which the knee joint bends around the knee axis after the heel strike, if appropriate against a resistance force via a hydraulic damper. At the end of the swing phase, the artificial knee joint can be stopped at a certain knee angle via an extension stop, in order to initiate or contribute to the initiation of the stance phase flexion. The setting of an extension stop in such a way that there is no fully extended leg at the initial heel strike at the end of the swing phase, i.e. the maximum knee angle allowed by the design is not set, and instead the achievable knee angle is reduced, is referred to as preflexion and has a positive effect on the gait behavior, since smoother walking is made possible.

Typical values of the extension stop for walking on level ground are a knee angle of around 176°.

In walking situations that deviate from walking on level ground, a control that is adapted for walking on level ground is often not sufficient and hinders the user in such special situations.

The object of the present invention is to make available a method by which a user of an artificial knee joint is better able to master special situations that arise during walking.

According to the invention, this object is achieved by a method having the features of the main claim. Advantageous embodiments and developments of the invention are disclosed in the dependent claims, the description and the figures.

In the method according to the invention for controlling an artificial knee joint comprising an upper part with an anterior face and a posterior face, a lower part which is mounted on the upper part so as to be pivotable about a knee axis and has an anterior face, a foot part arranged on the lower part, at least one sensor, a control device connected to the at least one sensor, and an actuator which is coupled to the control device and via which an achievable knee angle between the posterior face of the upper part and the posterior face of the lower part at the end of the swing phase can be set, provision is made that, on the basis of sensor data from the at least one sensor, it is concluded that a height difference of the foot part relative to a foot or a foot part of the contralateral side of a patient in their stance phase, or relative to the immediately preceding stance phase of the foot part, is to be negotiated, and the knee angle achievable in the swing phase is adjusted, particularly as a function of the determined or estimated height difference, preferably in the swing phase. The achievable knee angle, in particular the knee angle achievable in the swing phase extension, differs from the knee angle maximally provided by the design in that it is set by the actuator and is variable, whereas the knee angle maximally provided by the design generally signifies a maximally extended leg with a knee angle of 180°. The knee angle maximally provided by the design is predefined by the configuration and arrangement of components of the artificial knee joint.

Negotiating a height difference can involve, on the one hand, walking up an incline or climbing stairs or, on the other hand, negotiating a physical difference in height. However, it is also possible that it involves the intention of the user to position the foot accordingly, without there being a physical incline or height difference present. Climbing stairs can involve negotiating one or more steps and/or levels. That is to say, it can involve stepping onto a higher level, for example negotiating a curb, or climbing a staircase, i.e. several successive steps.

The setting of an extension stop in such a way that there is no fully extended leg at an initial contact, e.g. at the initial heel strike, at the end of the swing phase, i.e. the maximum knee angle allowed by the design is not set, and instead the achievable knee angle is reduced, is referred to as preflexion and has a positive effect on the gait behavior, since smoother walking is made possible. Walking up an incline or climbing stairs, or otherwise negotiating a height difference, by the user of the artificial knee joint, differs from the walking behavior on level ground. When walking normally on level ground, the contralateral foot and ipsilateral foot are at the same vertical distance from the hip during the initial set-down. By contrast, when negotiating a height difference, the vertical distance of the leading foot from the hip must shorten in order to compensate for the height difference. In the physiological gait, this is done by initiating a strongly pronounced hip flexion on the side of the leading leg and setting the leg down in an accordingly flexed position. Furthermore, especially with quite large height differences and small stride lengths, the center of gravity of the body is initially left above the supporting leg, and the weight is transferred only when the leading foot is set down. When walking on level ground, the stride lengths of the aided leg, i.e. the leg fitted with an artificial knee joint, be it with a prosthesis, an orthosis or an exoskeleton, are the same as those of the unaided leg. The center of gravity of the body moves substantially evenly between the supporting leg and the swing leg when walking on level ground.

When the user intends walking up an incline or climbing stairs or otherwise negotiating a height difference while walking, the center of gravity or the pelvis does not move forward evenly; instead, the user of the artificial knee joint stands on the back leg, which hardly contributes to the heel strike. The almost complete heel strike takes place via the leg in the swing phase, i.e. the leg that is lifted and is placed or intended to be placed at a higher level than the supporting leg. The achievable knee angle of the artificial knee joint is dependent on a determined or estimated height difference of the foot part of the aided side, i.e. the side provided with the artificial knee joint, to a foot or a foot part of the contralateral side of a patient in the stance phase. It has been shown that when the preflexion is greater than when walking on level ground and a lower knee angle is thus achievable, walking up an incline and climbing stairs is made much easier. In contrast to sitting down with the knee joint extended or only slightly forwardly flexed, the horizontal lever arm between the force application point on the foot and the hip is shortened with the same height difference when sitting down with the joint more strongly forwardly flexed, and moreover the necessary hip extension moment around the center of gravity of the body is reduced via the aided leg. The center of gravity of the body does not have to be levered over the entire, extended leg length, but instead only by a smaller lever due to the shortened leg length. This makes it possible overall to achieve a more harmonious movement when walking up an incline or climbing stairs. Loading compensation mechanisms, such as increased plantar flexion of the foot of the supporting leg or increased forward inclination of the upper body, can be reduced. The stride length is in a better proportion to the contralateral side, as a result of which the gait pattern becomes more symmetrical and more natural. With passive feet and foot parts, the foot is also set down on the ground in a more favorable orientation. Advantageously, the achievable knee angle is reduced by 5°-30° compared to the angle maximally provided by the design. Particularly in connection with a movable ankle joint, for example an adaptation of the ankle angle to the slope of the ground, but also with active support of the extension movement in the stance phase, it can be advantageous to reduce the achievable knee angle beyond this range, especially when negotiating particularly large differences in height.

Walking up an incline, climbing stairs or otherwise negotiating a height difference as intended by the user can be deduced from a determined or estimated height difference. The height difference between the foot or foot part of the contralateral leg and the leading foot or foot part in the swing phase is preferably used for the control. Another possibility is to use the height difference that the ipsilateral foot overcomes in the swing phase.

The achievable knee angle, in particular the knee angle achievable in the swing phase extension, can in particular be adapted during the swing phase of a stride. The adaptation is therefore preferably carried out in such a way that the achievable knee angle is matched to the subsequent initial contact and/or the subsequent stance phase. However, it is also possible that the achievable knee angle is already adjusted in the previous stance phase or a previous stride, in particular that walking up an incline, climbing stairs and/or the intention to negotiate a height difference was recognized in the previous stride and, on the basis of this information, the achievable knee angle is adjusted for the following stride. It is also possible, for example, that the achievable knee angle is adjusted only when walking up an incline, climbing stairs and/or the intention to negotiate a height difference is recognized for several successive strides. Furthermore, it is possible that the achievable knee angle remains unchanged over several successive strides, for example if several successive upward strides are taken and an adjustment only takes place when a different situation is detected.

In the terminal stance phase, knee flexion with low flexion resistance can be permitted and/or knee flexion can be initiated, in particular in accordance with sensor data, which allow conclusions to be drawn concerning the negotiating of a height difference, wherein the knee angle achievable in the swing phase is adjusted.

In a further development of the invention, provision is made that the achievable knee angle is reduced in the event of ascending movement, that is to say a height difference increasing counter to the direction of gravity. The greater the height difference between the foot of the supporting leg and that of the free leg, the smaller the achievable knee angle that is set. In the case of quite a high step or quite steep terrain, the extension is therefore stopped earlier. Conversely, this means that, in the case of shallow steps or a slight incline, the achievable knee angle is reduced to a lesser extent, thereby facilitating forward movement. The adaptation of the achievable knee angle to the height difference can take place continuously and/or in several discrete steps. It is also possible that the achievable knee angle is not reduced any further from a certain height difference. In particular, by adapting the knee angle to the height difference and/or the stride height, it is possible to reduce the load on the aided or ipsilateral side of the user of the artificial knee joint, who does not usually have full functionality of the leg muscles.

The height difference to be negotiated, as a parameter for the achievable knee angle, can be detected and/or determined via the trajectories of the hip joint, the knee axis and/or the foot part of the respective ipsilateral side. A trajectory describes the time profile of the position of a point in space. The translational path of a point which is connected to the artificial joint and is positioned, for example, on the upper part or the lower part or on the knee axis, and thus also the vertical component, can for example be defined from its determined acceleration values by means of double integration. The initial conditions of the integration are determined, for example, using a kinematic model, wherein the start of the integration advantageously lies in the late stance phase. The segment lengths required for the kinematic model can be measured and stored in the control device that is required to calculate the control signals for the actuator. A kinematic chain allows conclusions to be drawn, from the trajectory of one point via the relative degrees of freedom and segment lengths, concerning that of another point, for example that of the hip, the knee axis or the foot part. The degrees of freedom and segment lengths are known or stored in the control device, such that no movement data or other data of the unaided, contralateral leg have to be used for the determination. For example, the acceleration and the orientation of the lower part are determined via an inertial sensor, the angle between the lower part and upper part is determined via a knee angle sensor, and the trajectory, the speeds and accelerations of the hip are determined by integration of the acceleration data and the kinematic chain. The speeds and accelerations, preferably the vertical components, can in particular be used as indicators of negotiating a height difference. When negotiating a height difference, on the one hand the center of gravity of the body and thus the hips are raised. On the other hand, the knee is moved particularly quickly forward and upward. Alternatively or in addition, the distance covered, the speed and/or the acceleration of one or more points, in particular of the lower part and/or the knee axis, can be used, in particular the ratio of a horizontal and a vertical component, in order to conclude that a height difference is being negotiated.

Since it can be assumed that, in the stance phase on the contralateral side, when the foot is on the ground, the speed there is 0, in particular the horizontal speed component, the trajectory of the hip can alternatively or additionally be determined from one or more angle measurements on the contralateral side and known segment lengths. The entire hip advancement and hip elevation can then be calculated using an angle measurement and the known leg length of the contralateral side.

The height difference between the contralateral foot in the stance phase and the ipsilateral foot or foot part in the swing phase can be calculated or estimated using the vertical path of the hip joint of the aided, ipsilateral leg, the vertical path of the knee axis and/or the vertical path of the foot part and can serve as a parameter for the achievable knee angle. The vertical path of the hip joint of the aided leg can be determined, for example, from determined acceleration values of a point that is fixedly connected to the artificial knee joint, for example on the upper part or the lower part or on the knee axis, as has been described above. The trajectory of this point can be determined by double integration. Using a kinematic chain, the trajectory of the hip joint can be determined therefrom as a function of the relative degrees of freedom and segment lengths. The degrees of freedom and segment lengths are known, stored and available in the control device, such that the vertical path of the hip joint can be calculated therefrom without the need to use movement data or other data from the unaided, contralateral leg.

The vertical path of the knee axis can be determined, as described above, by double integration of the accelerations of a fixed point on the artificial knee joint or components arranged thereon, for example a prosthesis socket; the same applies to the vertical path of the foot part.

The movement of the hip and/or of the trunk can also be determined directly via a sensor which is attached to the hip or to the trunk, for example an inertial sensor which detects accelerations. From the accelerations, it is possible to calculate speeds and trajectories by double integration.

In a development of the invention, provision is made that, as the parameter for the achievable knee angle, the height difference is determined via a hip angle of the aided leg or the spatial orientation of the upper part and possibly its time profile. For the spatial orientation of the upper part, an inertial angle sensor can be arranged on the upper part, such that a direct measurement of the spatial position of the upper part is possible. For example, an inertial angle sensor or an IMU (inertial measurement unit) can be arranged in or on the artificial knee joint. A knee angle sensor is usually also arranged on the prosthetic knee joint or another artificial knee joint, such that the spatial orientation of the upper part can be calculated together from the spatial orientation of the lower part and the knee angle or hip angle. The spatial orientation is the alignment to a substantially unchangeable reference direction, for example the direction of gravity or a horizontal. To do this, no sensors are required on the contralateral, unaided side of the patient.

The hip angle can be measured directly as the relative angle between the trunk and the upper part or thigh. Alternatively, the spatial orientation of the trunk can be assumed or measured by means of an IMU and the hip angle determined together with the orientation of the upper part or thigh. In particular, the symmetry of the profiles of the hip angle and/or of the orientation of the upper part with respect to the vertical neutral position, for example as a ratio or difference, a swept angular range and/or a high flexion speed can be used as indicators for the detection and/or the determination of the height difference that is to be overcome. A height difference counter to the force of gravity can be assumed if the upper part is guided into strong flexion, a large angular range is swept over and/or a particularly rapid hip flexion takes place. The thresholds and sizes for the detection can be related to the walking speed in order to distinguish the influence of the walking speed on the temporal angular profiles from that of the height difference that is to be negotiated.

In a development of the method, provision is made that the height difference to be negotiated between the aided leg and the unaided leg is detected, calculated and/or estimated from the ratio of a translational, horizontal movement of the hip joint of the aided leg or the knee axis to the hip angle or the spatial orientation of the upper part. To calculate the height difference, the translational movement of a point on the prosthesis or orthosis, for example the movement of the knee axis, can be calculated, for example by double integration of measured linear accelerations with suitable initial conditions, and also by absolute and relative angles of the kinematic chain as far as the hip. The initial conditions of the integration are determined via a kinematic model, wherein the start of the integration advantageously lies in the late stance phase. In the case of an assumed purely rigid body movement, for example, the rollover point on the foot part and its profile over time can also be formulated as a function of loading and position or location. The segment lengths required for the kinematic model can be measured and stored in the control device that is required for calculating the control signals for the actuator. With the translational movement of the hip or the horizontal movement of the hip joint, it is possible to evaluate the movement and to draw conclusions concerning the gait behavior and the walking situation. The horizontal component of hip movement represents the proportion of a forward progression that is generated via the supporting leg. The hip angle or the orientation of the upper part controls the positioning of the swing leg side. Both aspects of the movement are coordinated with each other and are therefore suitable for detecting whether it is a case of walking up a slope or climbing stairs. By virtue of the coordination of the movement of both the supporting leg and the swing leg, conclusions can be drawn concerning the swing leg movement that is to be achieved for the aided leg. If the upper part can be brought into flexion particularly strongly or quickly in relation to the horizontal movement of the hip, it can be concluded that a height difference is to be negotiated counter to the force of gravity. Alternatively, the relations of horizontal hip movement and horizontal knee axis movement, as well as those of horizontal knee axis movement and orientation of the upper part or hip angle can be used. All variables can be derived entirely from sensor data of the aided side.

In a development of the invention, provision is made that the height difference is determined or estimated from a determined knee angle, for example by direct measurement via a knee angle sensor, and/or from the ratio of the spatial orientation of the upper part and/or lower part or thigh and/or lower leg. If the hip angle is available, it can be used to calculate the height difference. The hip angle can either be calculated or estimated by the IMU using an assumed orientation of the upper body and the determined spatial orientation of the upper part or thigh, or determined from the IMU using a spatial position sensor on the upper body, for example on an orthosis or an exoskeleton, in combination with the upper part orientation. The height difference can be determined or estimated from the time profile, from the ratio of the knee angle to the orientation of the upper and/or lower part and/or from the ratio of the orientation of upper and lower part to each other. The time profile and the movement of the segments in relation to one another, for example a particularly fast, pronounced or sustained bending or swinging upward, provide information on the intention of the user and the height difference that is to be negotiated. It is thus possible to detect in the swing phase whether the user is walking up a slope, climbing stairs or otherwise negotiating a height difference, such that the achievable knee angle, in particular in the swing phase, is determined and set.

The achievable knee angle can be set using an adjustable mechanical extension stop. The mechanical stop can be adjusted via various actuators, for example via a motor-driven end stop, by rotating an eccentric, by longitudinal displacement of a stop, by stiffening a buffer or in some other way. It is likewise possible to adjust the extension stop hydraulically or pneumatically by closing a valve depending on the achieved knee angle, such that no fluid can flow from an extension chamber into a flexion chamber or a compensation tank. It is likewise possible to stiffen the extension stop by stiffening a cushion, for example by filling a stop buffer with hydraulic fluid or pneumatic fluid. The stop can be formed by locking a drive, for example a motor, the adjustment being made by locking the motor after the desired knee angle has been achieved. Alternatively, the extension stop can be adjusted by a magnetorheological fluid and activation or deactivation of a magnetic field. When using functional electrical stimulation, stopping can be obtained by activation of the knee-flexing muscles. In all of the methods mentioned, it is not absolutely necessary to effect a physical blockage in the extension direction. It is sufficient to bring the extension movement to a standstill at and/or before the desired knee angle and/or to slow it down in such a way that the achievable knee angle is not exceeded, for example by predictive control. By means of said actuators, it is also possible to control the resistance of the knee joint against bending or stretching in order to achieve a controlled extension and/or flexion movement. Furthermore, it is possible to actively extend and/or flex the joint by means of an actuator, for example a motor, a pump, a spring, a spring accumulator, by electrical stimulation or by some other actuator which can generate a movement counter to a force, in particular to achieve the desired amount of knee flexion at the end of the swing phase.

In a development of the invention, provision is made that a further parameter used for the achievable knee angle is the spatial orientation of the lower part. During physiological climbing of stairs or when walking up an incline, the lower leg remains in a comparatively narrow angular range with respect to the vertical at the end of the swing phase and upon initial contact. The achievable knee angle can therefore be adapted such that, when walking up an incline or climbing stairs or negotiating an obstacle or a height difference, a defined orientation of the lower part is achieved at the end of the swing phase and/or upon initial contact. In addition, from the orientation of the lower leg upon initial contact, it is possible to draw a conclusion as to whether walking up an incline or climbing stairs or negotiating an obstacle or a height difference is taking place. While the spatial orientation of the upper part at initial contact depends on the stride height that is to be achieved, the spatial orientation of the lower part varies only slightly. On the basis of the determined stride height, a desired orientation of the lower part at initial contact can be predefined, and the corresponding achievable knee angle can be calculated according to the orientation of the upper part.

The desired orientation of the lower part can depend not only on the stride height but also on the walking speed and/or the stride length. With the walking speed and the stride length, the hip moments introduced by the user change, as do the heel strike of the leading leg, the stride duration, and also the force application point on the prosthetic foot or foot part and/or its time profile. It is therefore advantageous to adapt the achievable knee angle accordingly. In particular, it is advantageous to reduce the achievable knee angle at slower walking speed. The walking speed and also the stride length can be determined via sensor data, in particular via inertial sensors, which detect the spatial orientation of segments, and their change over time, and also accelerations. Speeds and position can be determined from accelerations through integration. The stride length can in particular be derived from the horizontal movement of the hip and/or of the knee axis. Alternatively or in addition, the stride length can be derived from the forward inclination of the aided leg at the end of the terminal stance phase.

In a further development of the method, provision is made that the height difference is determined or estimated from a knee angle, measured with a knee angle sensor on the artificial knee joint, and from a spatial position of the upper part or lower part, measured by a spatial position sensor arranged on the artificial knee joint. It is thereby possible to detect in the swing phase whether the user is walking up an incline or climbing stairs, such that the increased preflexion and the reduced achievable knee angle are already determined and set in the swing phase. The height difference can be determined from three parameters, namely the knee angle, the spatial orientation of the upper part and the spatial orientation of the lower part. Alternatively, the height difference is determined from two of the three parameters, for example from the two spatial orientations or from the knee angle in connection with a spatial orientation of either the upper part or the lower part.

In a further development of the invention, provision is made that the achievable knee angle is set in the swing phase of the aided, ipsilateral leg and is maintained until a predetermined spatial position and/or movement of the lower part and/or the upper part, a predetermined rotation and/or rotational speed of the lower part and/or upper part in space, an ankle joint angle, a predetermined force application point in the foot part, a predetermined force on the foot part, a defined moment on the foot part, the knee axis or the hip axis, the position of the ground reaction force vector, a defined acceleration on the foot part and/or a specified period of time is reached. Only after reaching, for example, a predetermined spatial position or rotation of the lower part and thus of the lower leg, in particular after the movement reversal of the lower leg and/or a sufficient reduction in the rearward inclination of the lower leg compared to the end of the swing phase, can it be concluded that a sufficient loading and rollover movement has taken place, such that the achievable knee angle can be increased and the knee joint can be extended further. For example, maintaining the maximum knee angle can be controlled when the spatial position of the lower part or lower leg changes after contact with the ground. If a forward rotation of the lower part or lower leg by a defined angle, for example by 5°, is detected after the foot part contacts the ground, for example via the spatial position sensor, the locking of the knee joint can be canceled and an extension permitted.

The same applies to the upper part, which at the end of a hip flexion phase achieves a defined spatial orientation at the end of a stride cycle when walking up an incline or climbing stairs. Using an ankle joint angle, data can be obtained concerning the rolling behavior, the local slope of the ground and the positioning of the center of gravity over the ankle joint, from which it is possible to draw conclusions regarding the stride profile. Instead of an angle sensor, force sensors can be arranged on or in the foot part and lower part, which force sensors determine the position of a force application point in the foot part and the position and extent of the ground reaction force. Through the profile of the force application and ground reaction force from the heel strike to the forefoot loading, or in the course of the rolling movement at initial contact on the forefoot, it is possible to determine or estimate the progress and thus the respectively adopted phase of the movement. The impact of the initial contact can be determined via an acceleration sensor on the foot part and/or lower part, and a conclusion can thus be drawn as regards the set-down of the foot. Furthermore, a forward movement to be achieved can be deduced from the hip extension moment, especially an extending moment, and an extension of the knee can be permitted. In particular, it is possible for the reduced knee angle to be maintained in a phase of the load transfer and/or early rolling phase.

Alternatively or in addition, the extension stop can be changed after a predetermined time, in order to provide increased safety through an increased extension movement of the knee joint. It is to be assumed that, after a certain period of time, either a progression of movement or a change in the movement pattern has taken place, such that increased safety through an extended knee joint is desirable. For example, the user of the artificial knee joint can stop on a step or pause while walking up an incline, for which purpose a knee joint that is extended to the maximum is advantageous.

In a further development of the invention, provision is made that a knee extension movement is permitted in the stance phase following the swing phase. The extension movement can be controlled according to the knee angle and/or the knee angle speed, the spatial orientation of the upper and/or lower part, the ankle angle, and/or the position, location and extent of the ground reaction force. An extension resistance can be set that is constant over the knee extension movement or that is coupled to the knee angle. The level and the profile of the extension resistance can depend on the stride height, the stride length, the walking speed, the knee flexion and/or the force application point on the foot when the foot is set down and/or the local slope of the ground. The resistance to the extension of the knee can increase degressively, linearly or progressively, in particular in the course of the rolling and knee extension movement. The extension movement can also be controlled such that the knee extension speed is controlled, in particular is kept constant or does not exceed a predefined value.

Alternatively, the extension movement can be controlled such that the lower part has an approximately constant orientation during the knee extension, and the thigh thus rolls over the knee axis, the rearward rotation of the lower part is limited or a defined forward rotation of the lower part is achieved. During physiological walking, there is typically a slight forward rotation of the lower leg. Due to the behavior of the foot or foot part deviating from physiological walking, for example the lack of possible dorsiflexion, it may make sense, in a deviation from the physiological gait, to achieve profiles for the movement of the lower part other than a slow forward rotation, for example an almost stationary lower part. The force application point on the foot can be determined via force sensors, and the extension movement can be controlled such that the force application point during the extension movement is controlled, preferably remains in the middle region of the foot and does not migrate in the direction of the heel or too early in the direction of the toes. A faster knee extension has the effect that the force application point migrates in the direction of the heel and less quickly in the direction of the forefoot; a slower knee extension causes the application point to shift in the direction of the forefoot. When setting the foot down with the forefoot, it is advantageous, on account of the larger lever arm of the ground reaction force around the knee axis, to provide a higher extension resistance than when setting it down with the heel. At a higher walking speed and/or a smaller knee angle when the foot is set down, it is useful to permit a faster extension of the knee joint, such that the foot performs an optimal rolling movement. By way of the ankle joint angle, it is possible to determine the local ground slope, on the basis of which the control of the extension movement can be adapted. The extension stop at the end of the stance phase extension is advantageously designed such that the extension movement is braked gently. In the case of an active knee joint, it is possible to actively support the extension movement. An interface enables the user to adapt the control parameters and thus influence the behavior in the stance phase extension himself. It is also possible that the extension behavior is adapted from stride to stride by the control, in order to adapt to the style of movement of the user, the characteristics of the foot part and/or of the shoe.

In a further development of the invention, provision is made that, after a minimum hip angle is reached and after a movement reversal of the thigh, i.e. after an increase in the hip angle, the spatial orientation of the lower part is kept constant until detection of an initial contact, of an axial force on the lower part and/or a change of an ankle joint angle. An initial contact can take place, for example, when the foot is set down on the ground or bumps against an object or an obstacle and can be detected by changes in the movement behavior, e.g. by detection of the acceleration behavior. When the foot part of the aided leg is lowered after a maximum hip flexion, it is possible, by adaptation of the extension resistances and flexion resistances or by an active system with drives, for the spatial orientation of the lower part to be kept constant, for example perpendicular or parallel to the vertical, e.g. until a set-down or rollover is detected. The set-down can be detected, for example, by the detection of an axial force or a moment on the lower part, accelerations of the lower part or by the time profile of the hip angle. A pause in the lowering movement can indicate that the foot has been set down and that the patient is being lifted to the next step via the aided leg. In addition to the orientation of the lower part, the spatial orientation of the connecting line from hip to foot or foot part (leg chord) can be controlled, in particular kept constant, after a minimum hip angle is reached, until detection of set-down of the foot. If a hip extension is carried out after the movement reversal of the thigh, the orientation of the leg chord can be kept constant, for example, by the knee joint being actively extended by an actuator. The knee angle can also be controlled in the course of the hip extension in such a way that the foot or the foot part maintains the same or approximately the same horizontal distance from the hip, that is to say the stride length is kept constant in a lowering movement.

It is also possible to control the knee angle, the orientation of the lower part and/or the orientation of the leg chord according to the translational hip movement, in particular the horizontal hip movement, preferably in order to bring the movement of the swing leg into a harmonious relationship to that of the supporting leg. For example, the knee angle can be increased if the hip is moved strongly in the anterior direction. It is also possible that a knee extension is achieved if, after reaching a first maximum hip flexion, the hip is again brought into strong flexion, i.e. the stride on the swing leg is continued forward in the late swing phase.

In a further development of the invention, provision is made that walking up an incline, climbing stairs or the like is detected via the time profile of the orientation of the upper part and/or the ratio of the upper part orientation to a translational, horizontal movement of the knee axis, and the achievable knee angle is adjusted on the basis of the profile and/or the relationship between the upper part orientation and the movement of the knee axis. The horizontal movement of the knee axis can be calculated from the known length of the upper part or thigh and from the profile of the orientation of the upper part over time together with the horizontal movement of the hip axis.

In a further development of the invention, provision is made that a flexion resistance in the swing phase of the aided leg after a reversal of the direction of movement of the lower part, that is to say the knee movement, is set to a level higher than when walking on level ground. In the swing phase of the aided leg, a flexion movement occurs first of all, i.e. a reduction in the knee angle. If the lower part or lower leg is then brought forward, i.e. the knee movement changes from flexion to extension after the knee axis has been raised to a higher level, it is advantageous to provide resistance to the flexion movement for safety reasons, for example in order to avoid stumbling in the event of bumping against an obstacle or a step, and in particular to prevent unwanted bending of the knee joint around the knee axis.

Advantageously, when walking up an incline or climbing stairs or the like is detected, the swing phase knee angle can be reduced by 5° to 20° in order to define the minimum achievable knee angle.

In a further development of the invention, provision is made that when walking up an incline or climbing stairs or the like is detected, the minimum achievable knee angle in the swing phase is reduced compared to when walking on level ground. When walking on level ground, the knee flexion is typically limited or reduced by a resistance in the direction of flexion, in order to achieve knee extension in good time at the end of the swing phase. On account of a smaller, minimum knee angle when walking up an incline, climbing stairs or the like, the lower part swings further up and approaches the upper part, as a result of which the ground clearance under the body is increased during the swing. To this end, the minimum knee angle is advantageously reduced when the height difference to be negotiated increases. Typical values for the reduction are from 5° to 20°.

In a further development of the invention, provision is made that, when walking up an incline, climbing stairs or when intending to negotiate a height difference in the stance phase, preferably in the terminal stance phase, a knee flexion is permitted with low flexion resistance and/or a knee flexion is initiated. The initiation of the knee flexion in the stance phase, thus when the foot is in contact with the ground, or under a load, corresponds to physiological walking in which the knee is flexed before the foot loses contact with the ground. The initiation of the knee flexion thus typically takes place during the rolling movement of the foot. The bending under a full or partial load at the end of the stance phase is known as the pre-swing phase. In order to permit simple bending of the knee joint, the resistance to movement in the bending direction is for this purpose reduced or kept at a low level in the stance phase, preferably in the terminal stance phase. Alternatively, in the case of active knee joints, a flexion movement can be initiated and/or supported under load. The reduction of the resistance to movement preferably takes place in the bending direction, or the initiation of the flexion movement is effected on the basis of sensor data. The achievable knee angle, in particular the knee angle that is achievable in the swing phase extension, is further adapted such that it supports walking up an incline, climbing stairs or negotiating a height difference in the following swing phase and/or in the subsequent stance phase. It is advantageous that the user can maintain the natural sequence of movement for initiating a knee flexion and a swing phase and does not have to perform a special sequence of movement in order to initiate swing phase flexion when walking up an incline, climbing stairs or negotiating a height difference.

In a further development of the invention, provision is made that the achievable knee angle when walking up an incline, climbing stairs or when intending to negotiate a height difference is set in the swing phase, wherein the user relieves the load on the prosthesis, the orthosis or the exoskeleton before initiating the knee flexion movement. The knee flexion can be initiated, for example, by the knee joint reducing the resistance to movement in the bending direction when the ipsilateral side is relieved and/or after it has been relieved and the user performs a hip flexion or a combination of a hip extension and a subsequent hip flexion. It is also possible that, in addition to the partial or full load, a further movement is necessary in order to reduce the movement resistance, for example a hip extension, in particular a rapid hip extension. A further possibility is that knee flexion is supported or actively initiated in the case of an active knee joint.

In a further development of the method, provision is made that the achievable knee angle can be set and/or changed over time, consciously and independently of the determined or estimated height difference. The user, for example an orthopedic technician, therapist or end user, can use an interface to set the control parameters. By inputting appropriate values or making appropriate adjustments, for example manually, the user can stipulate that the achievable knee angle should be increased and/or decreased. The setting by the user can be superposed on the other control parameters such that, for example, the control continues to set a lower achievable knee angle for larger height differences, but in both cases a greater achievable knee angle is set compared to the standard setting. It can also be made possible for the user to temporarily and completely deactivate the reduced achievable knee angle.

It is also possible for the system to adapt or determine parameters to control the achievable knee angle based on gait data, either through an ongoing, auto-adaptive adaptation or through a setting mode that is consciously activated and, after the setting has been made, deactivated again.

In a further development of the invention, provision is made that the height difference to be negotiated is detected and/or determined by determining the distance to the ground surface and/or determining the ground surface profile. The ground surface and/or the distance to the ground surface can be measured contactlessly, for example via sensors attached to the lower leg and/or foot part, in particular optically, using lidar, radar and/or infrared measurement, and/or by means of ultrasound measurement. From the measurement of several points on the ground surface, conclusions can be drawn regarding the ground surface profile and therefore the height of a height difference that is to be negotiated. Alternatively or in addition, the relative speed to the ground can be measured, in particular by utilizing the Doppler effect or by deriving a determined distance over time. The knee angle to be achieved, in particular the knee angle to be achieved at the end of the swing phase, is set according to the determined height difference.

In a further development of the invention, provision is made that the resistance to bending of the knee joint in the swing phase, in particular at the end of the swing phase, and/or during the stance phase, in particular during the initial contact and/or the load response, is set to a level higher than when walking on level ground. In the swing phase of the aided leg, a flexion movement occurs first of all, i.e. a reduction of the knee angle. If a knee extension then occurs after the knee axis has been raised to a higher level, it is advantageous to provide the flexion movement with a resistance that prevents unwanted bending of the knee joint about the knee axis. This is particularly advantageous in systems in which the resistance in the flexion direction and the resistance in the extension direction can be set independently of each other. Otherwise, the flexion resistance can be increased when the maximum knee angle is reached, when the foot is set down and/or when initial contact occurs, in particular to a level higher than when walking on level ground. The flexion resistance can be increased in such a way that bending of the knee joint is completely suppressed.

The flexion resistance at initial contact can also be configured such that a controlled knee flexion is permitted. The flexion resistance is adapted in particular in such a way that the flexion rate is controlled and/or the maximum flexion angle is limited by increasing the flexion resistance. The knee flexion can be controlled directly via a measured knee angle or via a measured spatial orientation of the lower part, such that the forward inclination of the lower part reaches or does not exceed a defined value. The level of resistance and the amount of permitted knee flexion can also depend on the height difference to be negotiated, on the walking speed, on the stride length and/or on the profile of the force application point on the foot during rollover, such that a maximum degree of safety and of support can be obtained for every situation.

Illustrative embodiments are explained in more detail below with reference to the accompanying figures, in which:

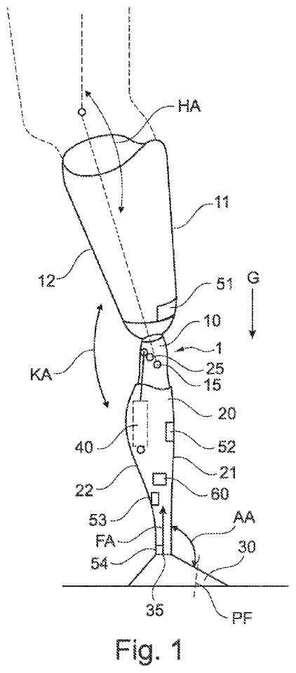

FIG. 1 shows a schematic representation of a prosthetic leg;

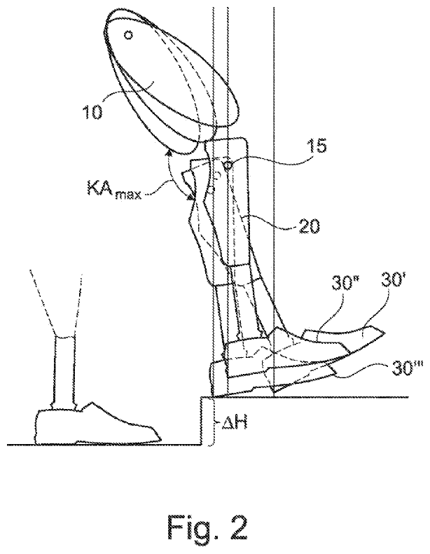

FIG. 2 shows a representation of different phases and situations when negotiating a height difference;

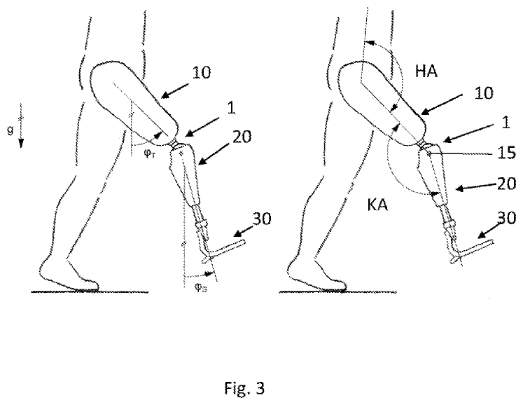

FIG. 3 shows a representation of a fitted prosthesis, with angles;

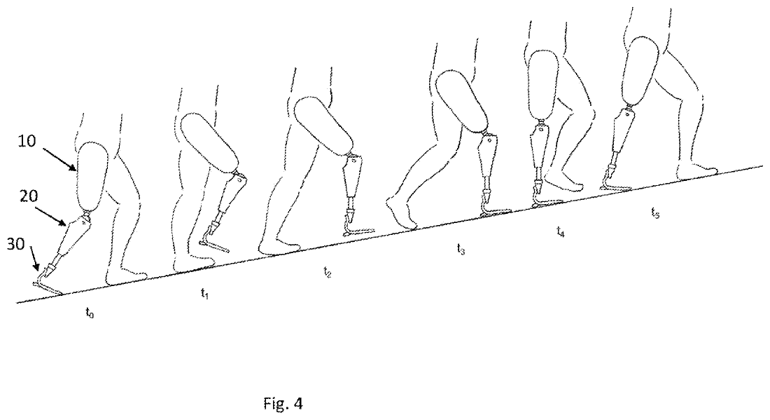

FIG. 4 shows a sequence diagram of walking up an incline;

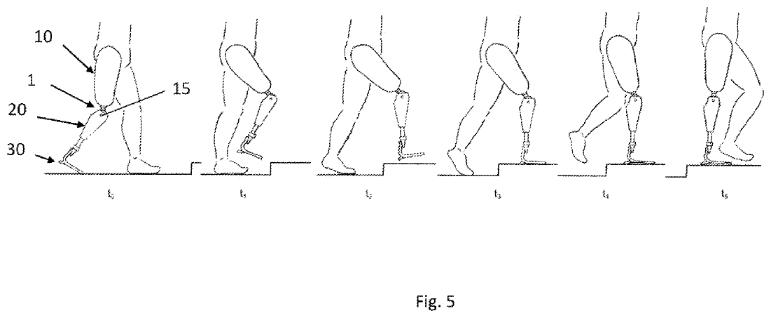

FIG. 5 shows a sequence diagram of negotiating a step;

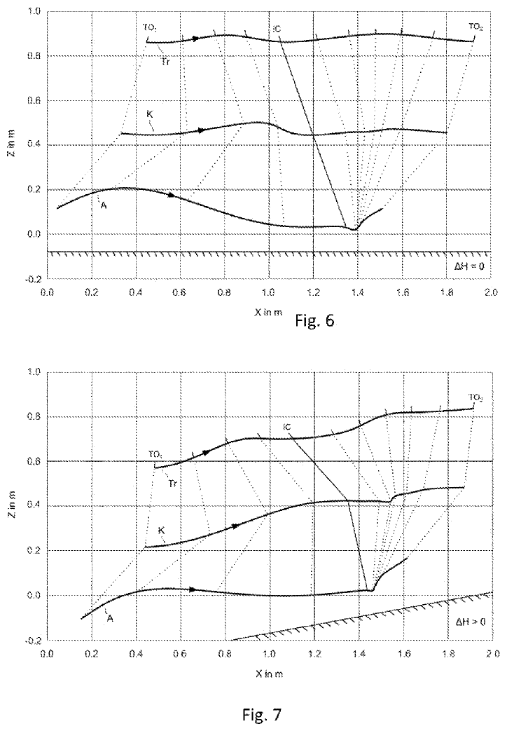

FIG. 6 shows trajectories of the ankle joint axis, the knee joint axis and the greater trochanter when walking on level ground;

FIG. 7 shows trajectories of the ankle joint axis, the knee joint axis and the greater trochanter when walking up an incline;

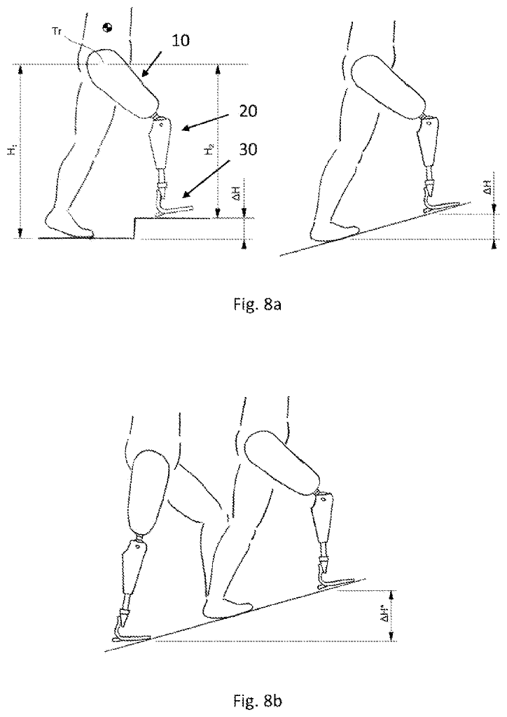

FIGS. 8a and 8b show representations of the height difference;

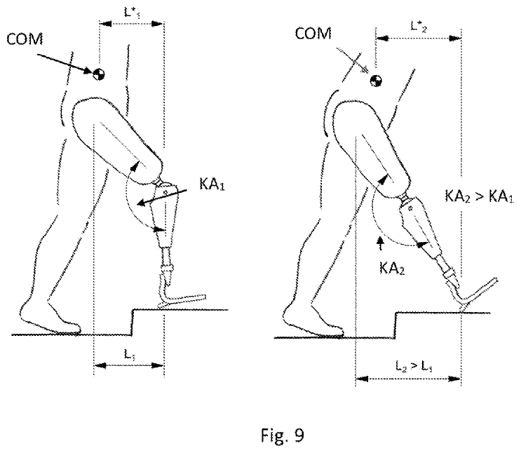

FIG. 9 shows representations of different heel strike situations;

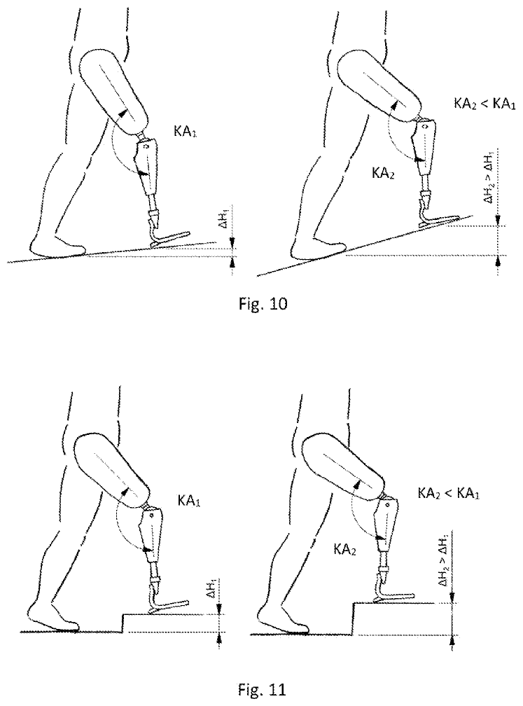

FIG. 10 shows the dependence of the knee angle on the height difference when walking up an incline;

FIG. 11 shows the dependence of the knee angle on the height difference when negotiating a step;

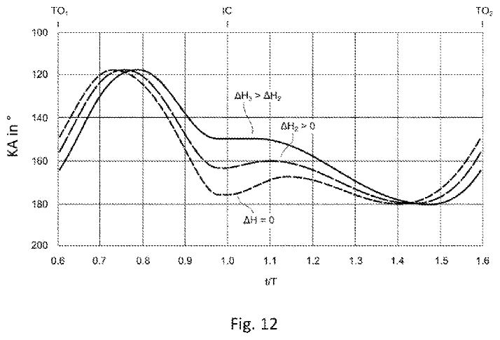

FIG. 12 shows knee angle profiles for different height differences over the relative time;

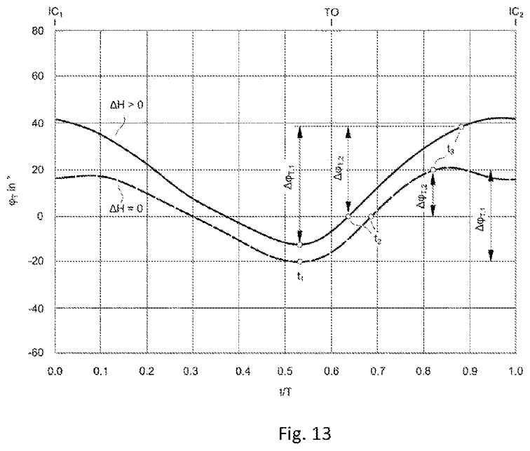

FIG. 13 shows the profile of a thigh orientation over a stride cycle;

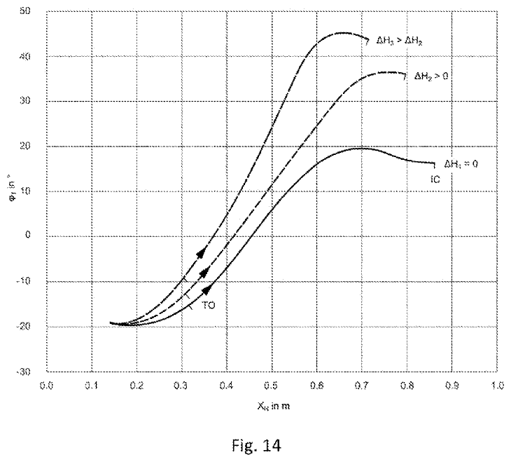

FIG. 14 shows the relationship of the thigh orientation in relation to the horizontal path of the hip;

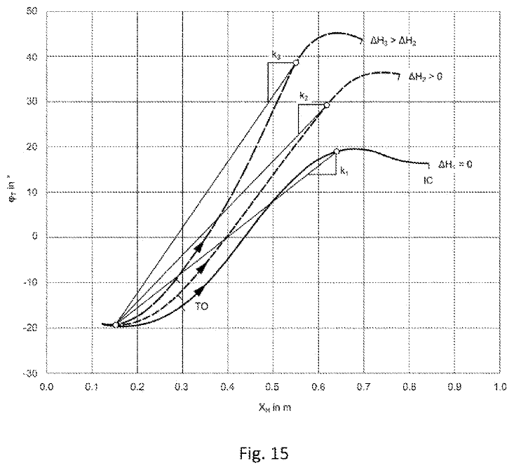

FIG. 15 shows a possible auxiliary variable for estimating the stride height;

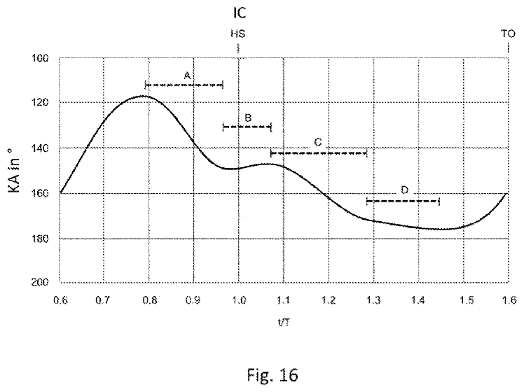

FIG. 16 shows the knee angle profile KA in over a stride cycle;

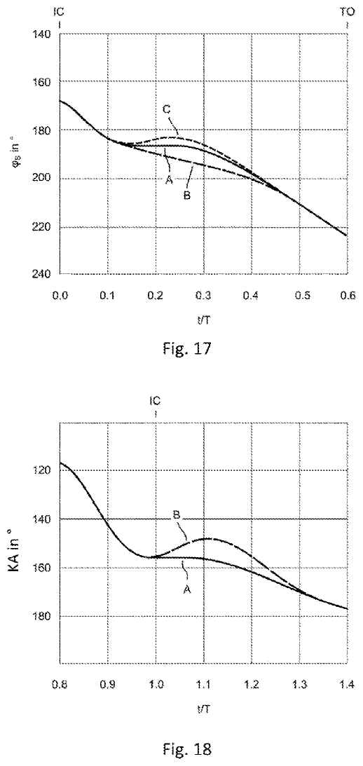

FIG. 17 shows different control profiles of a stance phase extension;

FIG. 18 shows two different knee angle profiles over phases of the gait cycle;

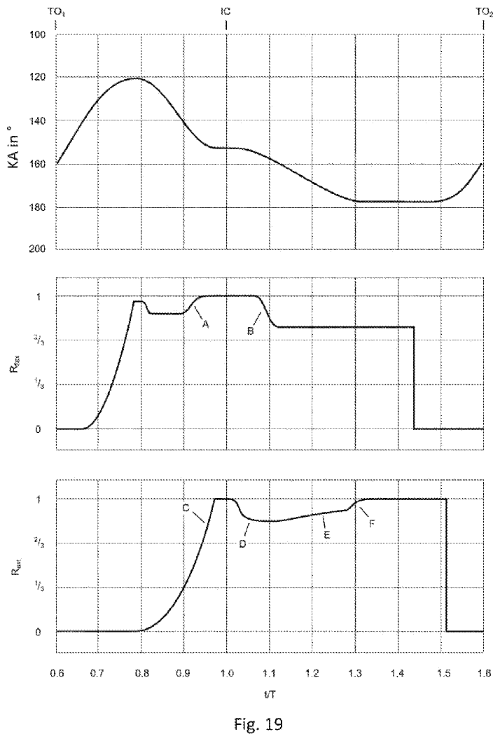

FIG. 19 shows a resistance profile in the case of passive control;

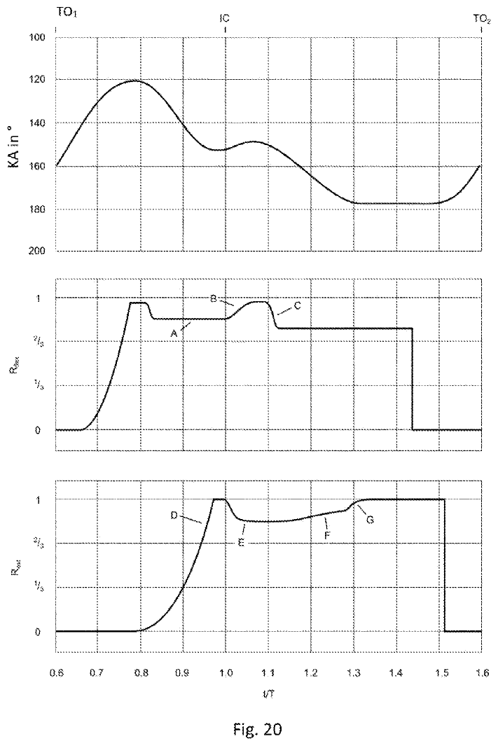

FIG. 20 shows a variant of FIG. 19;

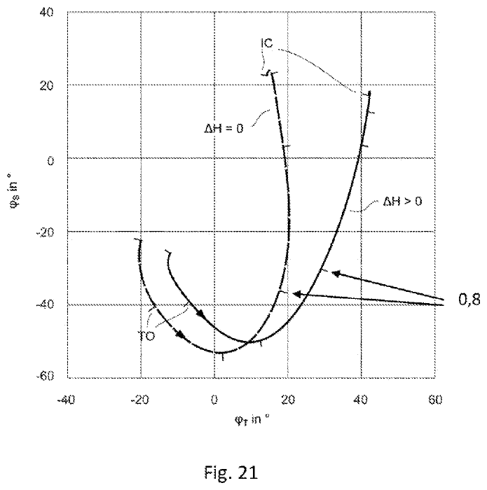

FIG. 21 shows the profile of a lower leg angle in relation to the thigh angle;

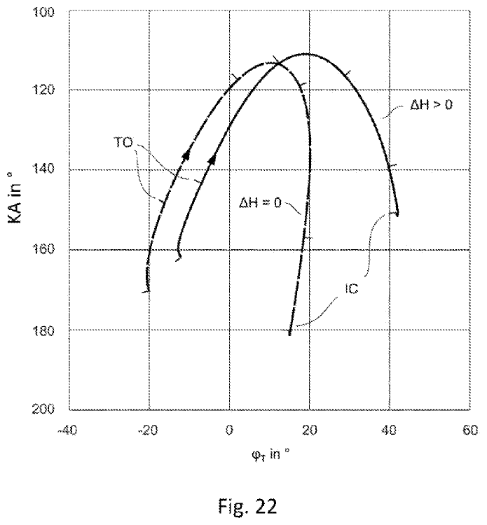

FIG. 22 shows the profile of the knee angle in relation to the thigh angle; and

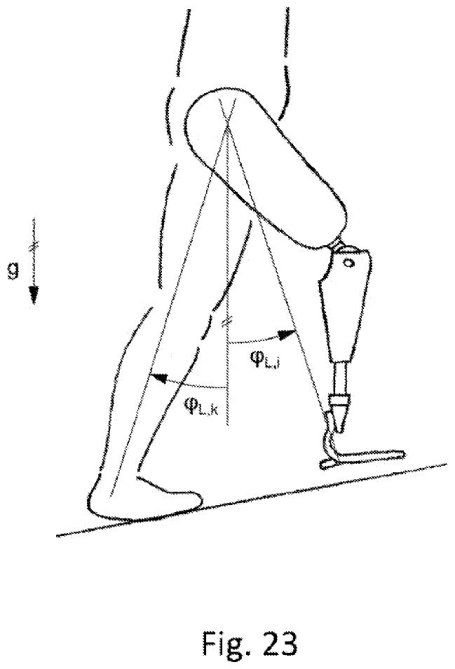

FIG. 23 shows a definition of the leg chords.

FIG. 1 shows a schematic representation of an artificial knee joint 1 in an application on a prosthetic leg. As an alternative to an application on a prosthetic leg, a correspondingly designed artificial knee joint 1 can also be used in an orthosis or an exoskeleton. Instead of replacing a natural joint, the respective artificial knee joint is then arranged medially and/or laterally on the natural joint. In the illustrative embodiment shown, the artificial knee joint 1 is in the form of a prosthetic knee joint having an upper part 10 with an anterior face 11, i.e. a front face in the walking direction, and a posterior face 12 lying opposite the anterior face 11. A lower part 20 is mounted on the upper part 10 so as to be pivotable about a pivot axis 15. The lower part 20 also has an anterior face 21 and a posterior face 22. In the illustrative embodiment shown, the knee joint 1 is designed as a monocentric knee joint. It is also possible in principle to control a polycentric knee joint correspondingly. At the distal end of the lower part 20, a foot part 30 is arranged which can be connected to the lower part either as a rigid foot part 30 with an immovable ankle or with a pivot axis 35, in order to permit a sequence of movement akin to the natural sequence of movement.

The knee angle KA is measured between the posterior face 12 of the upper part 10 and the posterior face 22 of the lower part 20. The knee angle KA can be measured directly via a knee angle sensor 25, which can be arranged in the region of the pivot axis 15. An inertial angle sensor 51 is arranged on the upper part 10 and measures the spatial position of the upper part 10, for example in relation to a constant force direction, for example the force of gravity G, which is directed vertically downward. An inertial angle sensor 52 is likewise arranged on the lower part 20 in order to determine the spatial position of the lower part during the use of the prosthetic leg.

In addition to the inertial angle sensor 53, a force sensor or moment sensor 54 can be arranged on the lower part 20 or the foot part 30, in order to determine an axial force FA acting on the lower part 20.

An actuator 40 is arranged between the upper part 10 and the lower part 20 in order to influence a pivoting movement of the lower part 20 relative to the upper part 10. The actuator 40 can be designed as a passive damper, as a drive or as a so-called semi-active actuator 40, with which it is possible to store kinetic energy and release the latter again at a later point in time in order to slow down or support movements. The actuator 40 can be designed as a linear or rotary actuator. The actuator 40 is connected to a control device 60, for example by a wired or a wireless connection, which in turn is coupled to at least one of the sensors 25, 51, 52, 53, 54. The control device 60 processes the signals, transmitted from the sensors, electronically using processors, arithmetic units or computers. It has an electrical power supply and at least one memory unit in which programs and data are stored and in which a working memory is available for processing data. After the sensor data have been processed, an activation or deactivation command is issued, with which the actuator 40 is activated or deactivated. By activation of the actuator 40, a valve can be opened or closed, for example, in order to change a damping behavior.

A prosthesis socket, which serves to receive a thigh stump, is secured on the upper part 10 of the prosthetic knee joint 1. The prosthetic leg is connected to the hip joint via the thigh stump. A hip angle HA is measured on the anterior face of the upper part 10, which angle is taken between a vertical line through the hip joint and the longitudinal extent of the upper part 10 and the connecting line between the hip joint and the knee joint axis 15 on the anterior face 11. If the thigh stump is raised and the hip joint flexed, the hip angle HA decreases, for example when sitting down. Conversely, the hip angle HA increases in the event of an extension, for example when standing up or performing similar movements.

During a gait cycle when walking on level ground, the foot part 30 is first set down with the heel; the first contact of the heel or of a heel part of the foot part 30 is called heel strike. Plantar flexion then takes place until the foot part 30 rests completely on the ground. As a rule, the longitudinal extent of the lower part 10 is then behind the vertical that runs through the ankle joint axis 35. While walking on level ground, the center of gravity of the body is then shifted forward, the lower part 20 pivots forward, the ankle angle AA decreases, and there is increasing loading of the forefoot. The ground reaction force vector migrates forward from the heel to the forefoot. At the end of the stance phase, toe-off takes place, followed by the swing phase in which the foot part 30, when walking on level ground, is shifted behind the center of gravity or the hip joint of the ipsilateral side while reducing the knee angle KA, in order then to be rotated forward after a minimum knee angle KA is reached, in order to then reach heel contact again with a generally maximally extended knee joint 1. The force application point PF thus migrates from the heel to the forefoot during the stance phase and is shown schematically in FIG. 1.

Walking on level ground differs from walking up an incline, climbing stairs or otherwise negotiating a height difference. The human gait is essentially defined by a coordinated movement of both legs. To take a stride, for example, the supporting leg has to take over the movement of the center of gravity of the body and generate forward progression, while the swing leg positions the contralateral foot in such a way that balance is maintained and an efficient weight transfer is possible.

The movement of both sides or of both legs is therefore functionally linked and can be observed during the most varied of movements. The functional linking of movements is simulated by modeling, and the functional linking of the components on the ipsilateral side and also on the contralateral side can be used to determine any missing information concerning individual segments from the behavior or the states of other segments. The method provides that the linking of the respective segments of the aided, ipsilateral side is used in order to simulate or control the leg movement and to recognize an intention and derive setpoint profiles and target values. The invention proposes, without a sensor system on the contralateral side, to analyze the movement and intended movement and to generate a control on the basis of this evaluation. While it is possible, with bilateral fittings, to obtain the movement of the respective contralateral side through sensors located on the prosthesis, orthosis or the exoskeleton or also via biosignals such as muscle activity or the like, this possibility is not available with unilateral fittings. Here, additional sensors would have to be arranged on the unaided, contralateral side, which would make the overall system much more complex. It is therefore proposed to use a model to determine the missing variables from the existing measured variables on the ipsilateral side, in order thereby to manage without instrumentation on the contralateral side. Even with sensors on the ipsilateral side alone, it is possible to obtain information about the movement of the contralateral leg in its stance phase, namely about the translational knee or hip movement, without these variables on the contralateral side being explicitly calculated. Sensors in the orthopedic device that receives the artificial knee joint record the states of the orthopedic device on the aided, ipsilateral side, and, optionally, individual variables on the contralateral side can be derived from these sensor values. With the aid of a model, the movement variables on the contralateral side are estimated from the measured data. In the case of a mechanical model, the boundary conditions and constraints can depend on the particular gait situation. For the control of the actuators, both the measured data, i.e. the sensor values, and the estimated variables are used to activate or deactivate the actuator.

The ipsilateral leg movement must be sufficiently determined from a technical point of view. For a cross-knee orthopedic device, for example, an inertial angle sensor 52 on the lower part, which records the absolute angle and the horizontal accelerations, and an angle sensor 25 for recording the knee angle KA between the upper part 10 and the lower part 20 are sufficient. To estimate the contralateral leg angle, for example, the ipsilateral leg movement is recorded, the hip translation is calculated from this, and a conclusion regarding the contralateral leg movement is drawn from the hip translation. For determining the translational movement of the hip, the translational movement of a point on the aided side, i.e. on the orthopedic device, for example the movement of the knee axis, is used. The translational movement of the knee axis, for example, is determined in particular via a double integration of measured linear accelerations with the appropriate initial conditions. In the further course of the process, the kinematic chain as far as the hip is followed using absolute angles and relative angles. The initial conditions of the integration can be determined using a kinematic model, wherein the start of the integration advantageously lies in the late stance phase. The roll-off point of the foot part, also called the center of rotation (COR), can be formulated as a function of load and position and included in the calculation. The segment lengths required for the calculation are measured and stored in the system, or assumptions are made based on statistical values. Since prosthetic fittings in particular are often custom-made, the individual segment lengths are known, since these necessarily have to be recorded in order to select components when assembling the prosthesis system. Alternatively, using anthropometric models, segment lengths can be determined with sufficient accuracy from characteristic lengths, e.g. the knee-ground dimension, or amputation features such as the amputation height, by means of scaling. Thus, from the measured acceleration of a fixed point of the orthopedic system, for example the position of the pivot axis, the trajectory of this point can be determined by double integration. By way of the kinematic chain, the hip trajectory is then defined as a function of the relative degrees of freedom and segment lengths. The translational movement of the hip is already a good measure for evaluating the intended movement; in particular, the horizontal component of the hip movement represents the proportion of the forward progression that is generated by the supporting leg. By virtue of the coordination of swing leg movement and supporting leg movement, the relationship of the ipsilateral swing leg movement to the hip translation permits a classification of the movement and a control of the prosthesis behavior. To identify which movement is being carried out or is intended, a combination of orientation of the upper part and hip translation or translation of the knee axis and hip translation is particularly suitable, since these variables can be determined entirely using the sensors in the orthopedic device.

In order to be able to estimate the leg angle of the contralateral side, wherein the leg angle between the hip joint and the set-down point at heel strike is measured in relation to the direction of gravity, two assumptions are made, namely that the contralateral foot is in contact with the ground and thus the relative movement between the foot and the ground surface is equal to 0, and that at least at one point in time in the double support phase, i.e. when both feet or foot parts are on the ground, an inertial leg angle of the contralateral side can be determined. One admissible assumption here would be that the leg angle on the contralateral side corresponds to the negative leg angle on the prosthesis side. Proceeding from this initial condition, the change in position of the contralateral leg angle can be calculated using trigonometric functions from the segment lengths and the relative translation of the hip. If the contralateral leg angle in its stance phase and the spatial orientation of the ipsilateral upper part in its swing phase are put in relation, the ratio can provide information on whether the user wishes to walk up an incline or climb stairs with the aided side or intends in some other way to negotiate a height difference ΔH when walking. Typical of such an intended gait behavior is a strong rearward inclination of the angle of the ipsilateral upper part at the middle of the swing phase, with a relatively low heel strike of the contralateral side in the stance phase. In other words, the contralateral side remains almost vertical, which means that the translational hip movement is small, while the upper part or the thigh is raised and flexed strongly.

If, when walking up an incline, the artificial knee joint 1 is stopped in the flexed position at the end of the swing phase, the extent of such a preflexion can be determined such that the ipsilateral and contralateral leg angles are in a harmonious relationship to each other when the aided side makes contact with the ground. The flexion and extension resistances in the form of setpoint values of the actuator 40 are then set in the orthopedic device in the swing phase such that a harmonious relationship is established between the leg angle on the contralateral side in the stance phase and the leg angle on the ipsilateral side in the swing phase. The setpoint values of the actuator 40 and thus also the flexion resistances and extension resistances are set such that the maximum achievable knee angle KAₘₐₓ is adjusted according to the determined or estimated height difference ΔH of the foot part on the ipsilateral side, wherein the height difference ΔH is applied to a foot or a foot part on the contralateral side of a patient.

If walking up an incline, climbing stairs or stepping over an obstacle by negotiating a height difference ΔH is detected, the maximum extension of the lower part 20 relative to the upper part 10 is limited, such that the maximum achievable knee angle KAₘₐₓ is reduced. The lower part 20 is stopped at a specific angle of the lower part 20. FIG. 2 illustrates such control using three states of an orthopedic device. If the lower part 20 were to be extended to the maximum extent when negotiating a height difference ΔH in a manner unchanged from walking on level ground, such that the achievable knee angle KAₘₐₓ is approximately 180°, the foot part 30′ would be set down very far forward and with a large sole angle, and the patient would have to rotate the hip about the set-down point over the entire leg chord length, which would lead to a non-physiological sequence of movements. According to the invention, by contrast, provision is made that the extension of the lower part 20 at a specific maximum knee angle or with a specific orientation of the lower part, which can be detected by the inertial angle sensor 52 for example, is stopped even before the maximum extension is reached, such that the foot part 30″ at the end of the extension movement is located above the ledge or step or at the end of the movement is located at the determined or estimated height difference ΔH. Subsequently, in the further course of movement, the thigh or the upper part 10 is lowered, wherein the orientation of the lower part 20 is preferably kept constant, i.e. the spatial position of the lower part 20 does not change, until the foot part 30′″ has touched the ground. This can be detected, for example, by the axial force sensor 54 detecting the occurrence of an axial force. If such an axial force FA is detected, it is to be assumed that the swing phase has ended and, in order to negotiate the height difference ΔH, both the hip angle HA is increased and the knee angle KA is increased, at least not decreased, so that, due to the variable knee angle setting and a preflexion, the effective leg chord length is shortened at set-down and less energy is required to negotiate the height difference ΔH.

The greater the height difference ΔH to be negotiated, which can be determined for example on the basis of a reduced heel strike on the contralateral side or a detected maximum spatial position of the upper part 10, the maximum achievable knee angle KAₘₐₓ is reduced, i.e. the preflexion is increased and the extension stop is shifted forward. The extension stop can be shifted forward by a motorized adjustment of a mechanical stop or by suitable opening and closing of valves in a hydraulic or pneumatic control system within the actuator 40.

The vertical path of the knee axis, i.e. the difference in height against the direction of gravity G, can be calculated from the absolute angle of the upper part 10, if the vertical path of the hip is known, or is determined as an estimate. The vertical path of the foot part can be calculated or estimated from a combination of the spatial orientation of the upper part 10 in conjunction with the relative angle or knee angle KA, which can be determined via the knee angle sensor 25. The knee angle sensor 25 allows the determined knee angle KAD to be determined and, if sensor data are available on the hip angle in conjunction with the segment lengths, serves to calculate the height difference ΔH. The achievable knee angle KAₘₐₓ is set in the swing phase of the ipsilateral leg and is maintained until a predetermined spatial position of the lower part and/or upper part is reached. Likewise, the setting with regard to the achievable knee angle KAₘₐₓ can be maintained, when monitoring the ankle joint angle AA, until a predetermined ankle joint angle AA is reached, which is defined for example as the angle that arises after the foot part 30 is lifted at the end of the stance phase, when the foot part 30 is in a neutral position. If the foot part 30 is then set down, the ankle joint angle AA changes, which is a sign that a change in the maximum achievable knee angle KAₘₐₓ is now possible. Alternatively, by determining the force profile along the longitudinal extent of the foot part, the position of the force application point can be determined and, depending on this position, the actuator 40 can be controlled accordingly in order to block further extension up to a certain point in time and only then to permit an extension of the knee joint 1. Alternatively or in addition, a timer element can be used to set a specific time period that limits a maximum extension.

Reaching a minimum hip angle HA can be detected by monitoring the spatial orientation of the upper part 10. If the thigh or the upper part 10 is maximally flexed, the longitudinal extent of the upper part 10 is at a maximum inclination relative to the direction of gravity G. If the upper part 10 is then pivoted downward about the hip joint and the longitudinal extent of the upper part 10 approaches the direction of gravity G, a minimum hip angle HA is reached and a movement reversal has taken place. After the detection of the movement reversal, the maximum knee angle or, for example, the spatial orientation of the lower part 20 can be kept constant until a set-down of the foot part 30 on the ground is determined, for example by detecting an axial force FA or by a change in the ankle joint angle KA. While the maximum achievable knee angle KAₘₐₓ is set by changing the extension resistance in order to set the foot part 30 down in the correct orientation with an angled leg, it is advantageous, for the further course of movement, if the flexion resistance in the swing phase of the ipsilateral side after a movement reversal of the lower part 20 in the vertical direction, i.e. when the lower part is lowered, is kept at a high level, at a level that is higher than the flexion resistance when walking on level ground, in order to facilitate lifting the body of the user of the orthopedic device when walking up an incline, climbing stairs or the like and to avoid unwanted flexion and bending of the knee joint 1.

In FIG. 3, the respective angles and spatial orientations and the respective reference values are shown in order to clarify the respective relationships among one another. The direction of gravity is denoted by the arrow g; the gravity orientation essentially corresponds to a vertical orientation. The spatial orientation of the upper part 10 is defined by the angle φ_{T}, the spatial orientation of the lower part 20 is represented by the angle φs, in each case measured from the direction of gravity g. The hip angle HA is measured between the longitudinal orientation of the trunk and the longitudinal orientation of the upper part 10 on the front side in the g direction; the knee angle KA is measured between the longitudinal extent of the upper part 10 and the longitudinal extent of the lower part 20 about the knee axis 15.

FIG. 4 is an illustration of a sequence of movement when walking up an incline. The sequence of movement begins at t₀ for the aided leg with the upper part 10, the lower part 20 and the prosthetic foot 30, in which the prosthetic foot 30 still just touches the ground and is at the end of the stance phase. The unaided, contralateral leg is fully placed on the ground and slightly flexed. At the time t₁, the aided leg is raised and is in a maximally flexed position with a minimal knee angle KA. At the time t₂, the foot part 30 is moved toward the ground and lowered, the lower part 20 is at the end of a swing phase extension movement and is braked, for example by activating a brake, increasing a damping rate or adjusting an extension stop, with which the achievable knee angle is changed. At the time t₃, the foot part 30 of the aided leg is set down with a flexed knee joint 1; the contralateral, unaided leg is unloaded and moved forward. At the same time, a stance phase extension is carried out for the aided leg, which is completed in the phase at the time t3. The center of gravity of the body is then moved forward in the walking direction via the knee pivot axis 15. With an extended knee joint, the lower part 20 performs a forward rotation about a support point or pivot point on the ground side and, in the illustrative embodiment, is arranged in the region of the tip of the prosthetic foot 30. The movement cycle then begins again.

FIG. 5 shows a corresponding sequence of movement for negotiating a step, wherein a further movement stage for negotiating a step is designated in FIG. 5 as t₄ and lies between the time segments and t4 in the sequence shown in FIG. 4. At the time t₄ in FIG. 5, the unaided, contralateral leg is raised and at a height just above the step to be negotiated, and the knee on the contralateral side has not yet been moved in front of the knee axis 15 of the prosthetic knee joint 1.

In FIG. 6, the trajectories of the ankle joint A at the height of the ankle joint axis 35, those of the knee K at the height of the knee joint axis 15 and those of the trochanter Tr, as a prominent point of the thigh bone in the region of the hip joint, are plotted. The orientations of upper part 10 and lower part 20 in the sagittal plane are shown between the trajectories shown in solid lines. The trajectories and likewise the orientations reflect walking on level ground; the arrow directions indicate the forward movement. At the start of the swing phase, at toe-off TO, the ankle joint A is slightly raised, compared to being straight during standing. After the toe-off, the knee joint K is brought forward and raised slightly, creating a whiplash effect in which the ankle joint A is raised and the greater trochanter remains at an almost unchanged level. With a further forward movement, the knee joint K is raised further and moved forward, the ankle joint A overtakes the knee joint after about 40% of the gait cycle until the knee joint K is in a maximally extended position, which is the case with heel contact or heel strike. This gait phase is marked with the solid line and the reference sign IC for initial contact. Due to the elasticity of the foot, the ankle joint axis sinks slightly and the leg rolls forward around the foot 30 or the ankle joint axis 35 in the walking direction, wherein the knee joint is slightly bent because this is a stance phase flexion. At about 70% of the gait cycle, the greater trochanter overtakes the knee joint axis, and the hip is brought in front of the knee joint and a forward movement is initiated. Each individual dashed line marks one tenth of a gait cycle.

FIG. 7 shows the trajectories of ankle A, knee K and greater trochanter Tr when walking up an incline, for example on a ramp. It can be seen from the different trajectories that the ankle joint A has the same trajectory shape, but that it is inclined upward. The lower leg orientation upon initial contact is different than when walking on level ground, as also is the orientation of lower part to upper part, namely bent in contrast to a maximally extended position when walking on level ground. All the trajectories end at a higher level than they began, which is the nature of things when walking up an incline.

On the basis of FIG. 8, the stride height between the contralateral, unaided leg and the ipsilateral foot part 30 of the aided leg can be defined. For example, the distance H₁ from the ground to a prominent point of the hip, for example the greater trochanter, is determined at the level of the supporting leg, the distance H₂ is the distance between the ground and the hip or the greater trochanter on the leading side, in the example shown on the aided side. The height difference HΔ then results from the difference between H1 and H2. Accordingly, a definition of the height difference ΔH applies for walking on a ramp. FIG. 8b shows the definition of a height difference ΔH* in which the negotiated height is measured from ipsilateral to ipsilateral, i.e. the height difference between the lifting of the aided leg and the setting back down, which corresponds to the height difference between the toe-off of the aided leg and the initial contact.

FIG. 9 illustrates the difference to the patient, when seeking to negotiate a height difference, between using the aided leg with a flexed knee joint and a leg with an extended knee joint. In the left-hand view, a preflexed configuration is shown, while the right-hand view shows an extended configuration in which the knee angle KA₂ is greater than in the preflexed configuration with a knee angle KA₁. Due to the preflexion, the heel strike L₁ is less than when setting down with an extended leg. The center of gravity COM of the body must be moved forward in order to achieve gait progress. To do this, the lever L_{*1} must be used as the distance between the center of mass COM and the vertical from the contact point, in order to move the center of gravity of the body. The lower the lever L_{*1}, the less the effort that the patient has to make via the thigh muscles and the hip extensor. In the view on the right, in which the heel strike is L2>L1, the lever L_{*2} is also much larger, even with a user leaning forward, so that considerably greater force is required to negotiate the height difference. In the case of an extended configuration as in the view on the right, the height difference LE must be achieved using a larger heel strike L₂ compared to a preflexed configuration. The usual compensation is effected by a forward inclination of the upper body, which attempts to reduce the lever L_{*} between the set-down point and the center of mass COM. In addition, there is an increased plantar flexion of the trailing supporting leg, which cannot be seen in the illustration.

FIG. 10 shows the dependence of the knee angle KA on the height difference ΔH or the stride height. The greater the stride height or the height difference ΔH to be negotiated, the smaller the knee angle KA becomes, in particular if the lower leg orientation is to be the same at set-down. FIG. 11 shows this relationship for negotiating a step; FIG. 10 shows it for walking up an incline on a ramp.

FIG. 12 shows the knee angle profile for different height differences ΔH. When walking on level ground, where ΔH=0, the toe-off TO₁ results in a reduction of knee angle KA down to a minimum knee angle. The foot is then brought forward, the knee angle KA increases until almost complete extension at heel strike or initial contact IC. A preflexion is set so that stance phase flexion can be performed. The stance phase flexion increases up to the point in time t/T=1.05 and then decreases until the maximum extension at t/T=1.4, which corresponds more or less to the rollover. Then, at the end of the stance phase, a preflexion is carried out to initiate the swing phase. With increasing height differences ΔH, it can be seen that the preflexion increases with the height difference ΔH in the event of a heel strike or initial contact IC; the stance phase flexion may decrease or be suppressed as the height difference ΔH increases. The knee angle KA is plotted over the dimensionless time by the proportion of the gait cycle; the subdivisions each correspond to 10% of a gait cycle.

FIG. 13 shows the profile of a thigh orientation ϕ_{T} in ° over a stride cycle with the subdivision into the respective proportion of the gait cycle, applied from a first initial contact or heel strike IC as far as a second initial contact IC2 or heel strike. The dashed line shows the profile of the thigh orientation φ_{T} for walking on level ground, the solid line for walking up an incline or climbing stairs with ΔH>0. In order to detect ascent, the height difference ΔH can be deduced from the profile of the thigh orientation φ_{T} on the basis of the greater range of motion or the greater pivoting in the period from the increase in hip flexion at T₁ to T₃, which is expressed in a greater Δφ_{T1}, from the greater hip flexion between T₂ and T₃ in the form of Δφ_{T2} or the ratio of hip extension to hip flexion

$$( \frac{φΔ T 2}{( φΔ T 1 - φΔ T 2 )} )$$

or the ratio of flexion to range of motion

$$( \frac{φΔ T 2}{φΔ T 1} ) .$$

The corresponding adjustment commands from the control device for adapting the damping and/or the stops then result from the calculation or estimation.

FIG. 14 shows the ratio of the thigh orientation φ_{T} in relation to the horizontal path of the hip or of the greater trochanter X_{H} for different height differences of the ΔH. Walking on level ground with ΔH1 results in a comparatively small range of motion; with increasing height difference ΔH, there is an increasing increase in the thigh orientation φ_{T} with a shortening stride length or a shortening horizontal path of the hip. From such a relationship, it can be deduced whether the person is stepping over an obstacle or walking up an incline and whether and to what extent an adjustment of the extension stop or damping devices should be undertaken. The adjustment of the extension stop or of the damper device can then take place in the swing phase, for example when a threshold value stored in the control unit for this ratio is reached.

FIG. 15 illustrates a possible auxiliary variable for estimating the stride height or the height difference ΔH to be negotiated, namely the ratio of the thigh orientation φ_{T} to the horizontal path of the hip X_{H}. A rising inclination K indicates an increasing stride height ΔH; the greater the stride height ΔH, the greater also is the inclination of the ratio of thigh orientation φ_{T} to a horizontal path X_{H} of the hip, for example of the greater trochanter.

FIG. 16 shows the knee angle profile KA in ° over a stride cycle, starting with toe-off TO, with a heel strike HS or initial contact IC at 1 and a second toe-off TO at 1.6. In the different gait phases, different goals are pursued via the control of the resistances or the stops. In region A, the swing phase extension is braked in a controlled manner or the knee joint is actively extended as far as the respectively desired preflexion angle. In phase B, the stance phase flexion is checked, for example bending under a high flexion resistance in order to limit or prevent excessive stance phase flexion. In phase C, the stance phase extension is influenced, for example via the extension rate, such that the rollover behavior and extension behavior can be influenced. In phase D, the stance phase extension is slowed down, in order to avoid a hard stop in the extension stop when the rollover has taken place and the maximum knee angle is reached.

An example of the application of an energy store, which can be integrated in an active or semi-active actuator, lies in the use of the energy store in selected gait phases. The kinetic energy can in particular be stored during the stance phase extension, that is to say during phases C and D, within these phases in particular during the braking in the stance phase extension, which corresponds to phase D. To support the swing phase flexion, especially directly after the initiation of the swing phase, the stored energy is released again. It is likewise possible that the kinetic energy is stored during the stance phase extension in phase D, in order to release it again during the swing phase extension in phase A, there in particular in the second half of the stance phase extension. The correct positioning of the foot is supported in this way. In principle, it is also possible to store the kinetic energy in other movement phases and to release it again in other movement phases.

It is not necessary for all of the stored kinetic energy to be released again immediately; amounts of stored energy can also be accumulated, for example over several movement phases of a stride or over several strides in different or in the same movement phases.

FIG. 17 shows different control profiles of a stance phase extension over the lower leg angle cps. The knee extension can be controlled in such a way that, with a profile according to A, the lower leg or the lower part 10 maintains an approximately constant orientation during the knee extension movement. Alternatively, according to profile B, a certain amount of forward rotation of the lower part 20 and the lower leg can be allowed, and a forward rotation speed can be set to a defined level. The profile C provides a certain amount of rearward rotation or a rearward rotation speed. All three control variants may depend on the walking speed, the stride height, the stride length and the degree of knee flexion. The lower leg angle cps is again plotted over the phases of a gait cycle from the initial contact IC to the beginning of the swing phase at toe-off TO.

FIG. 18 shows two different knee angle profiles KA, likewise over phases of the gait cycle, wherein the phase following the initial contact IC here takes place with a relatively rigid preflexion of 20 degrees. Stance phase flexion is suppressed over the profile according to the solid curve A. The profile with the dashed line B permits a further stance phase flexion to 30 degrees, but the extent of the stance phase flexion is monitored and the maximum knee flexion limited. Both variants can be used depending on the walking speed, the stride height, the stride length and the profile of the force application point in the foot part.

FIG. 19 shows the possible resistance profile with passive control and with suppression of stance phase flexion on the basis of three diagrams. The upper diagram shows the knee angle profile KA, the middle diagram shows the flexion resistance R_{flex} and the lower diagram shows the extension resistance Rₑₓₜ over a gait cycle from toe-off 1 to toe-off 2 with the initial contact IC or heel strike at 1.0. All three curves are plotted over the dimensionless time through the proportion of the gait cycle. Before the initial contact IC, the flexion resistance R_{flex} is increased to a maximum value, such that a maximum flexion resistance is applied at an initial contact IC of the foot part. The increase in phase A takes place during the swing phase extension, the knee joint is locked at initial contact IC. After the initial contact IC, the flexion resistance R_{flex} to the stance phase extension is reduced again in phase B, for example when a stance phase extension takes place, in order then to permit, at the end of the stance phase, a rapid drop in the extension resistance in order to initiate the swing phase. The extension resistance is increased in phase C before the initial contact IC during the swing phase extension, in order to stop the knee joint at a defined knee angle KA. There does not have to be a complete blocking of the extension movement. By increasing the resistance, the extension movement can be reduced sufficiently to adequately stop the joint. The extension resistance is then reduced, if necessary depending on the walking speed, stride height, stride length, existing knee flexion and the profile of the ground reaction force vector. The extension resistance Rₑₓₜ is then increased in a controlled manner during the stance phase extension movement, for example by regulating it to a setpoint extension rate of the knee joint or as a function of the lower leg angle φₛ. Finally, the stance phase extension is brought to a stop by a further increase in phase F, in order to avoid a hard stop in the extension or when the desired knee angle KA is reached.

FIG. 20 corresponds substantially to FIG. 19, but shows different profiles for both the knee angle KA and the respective resistances over the course of a gait cycle. In contrast to the profile in FIG. 19, the flexion resistance R_{flex} is not increased to a maximum value before the initial contact IC, but instead is reduced to a lower value, after a maximum for slowing down in the swing phase, until after the initial contact IC the flexion resistance R_{flex} is increased in phase B in order to permit a controlled stance phase flexion. The increase in phase B serves to control the flexion rate or the extent of the stance phase flexion. The extent to which the flexion resistance R_{flex} is increased depends on the desired maximum flexion angle. The flexion resistance is then reduced again in phase C, analogously to phase B in FIG. 19. The extension resistance Rₑₓₜ is adjusted as explained in the profile of FIG. 19.

FIG. 21 shows the profile of the lower leg angle φₛ in relation to the thigh angle φ_{T} for walking on level ground in the broken line with a height difference AR equal to 0. Here too, the distinctive points of the gait are marked with toe-off TO and the initial contact IC. The solid line shows the ratio of the lower leg angle φₛ to the thigh angle φ_{T} for walking up an incline or stepping over an obstacle with a height difference ΔH greater than 0. The subdivisions each correspond to 10 percent of a gait cycle. On the basis of the different profiles of the curves, it is possible to estimate how great is the height difference AH that has been overcome or is to be overcome. In particular, from the curve profile after 80 percent of the gait cycle, i.e. after two lines after the toe-off TO or at 0.8, there is a substantially steeper increase for walking on level ground with ΔH equal to 0 than for walking up an incline or stepping over an obstacle with a height difference ΔH greater than 0. For different height differences ΔH, different profiles can be determined or stored, which are then made available to the control device in order to be able to make a corresponding adjustment of the stops and the resistances for adaptation to the respective gait situation.

FIG. 22 shows the ratio of the knee angle KA to the thigh angle φ_{T} for walking on level ground with ΔH equal to 0 with the dashed line, and for stepping over an obstacle or walking up an incline with ΔH greater than 0 with the solid line. With a stride cycle of 0.6 at a toe-off, there is likewise a significant difference in the curve profiles in the range of 0.8 of a gait cycle, via which the height difference ΔH is then estimated using comparison algorithms, and a corresponding adaptation of the resistances or stops can be effected with the control.

In FIG. 23, a definition of the leg chords of an ipsilateral, aided leg and a contralateral, unaided leg is made. The leg chord goes through the hip rotation point and forms a line to the ankle joint. As can be seen from FIG. 23, the length of the leg chord and the orientation φ_{L} of the leg chords change during the movement, in particular also with different inclines. The height differences ΔH that are to be negotiated can be estimated and predicted or determined via the profile of the change in length and/or orientation of the leg chord. The respective control commands are then derived from this. The respective orientation of the ipsilateral leg chord φ_{Li} relative to the direction of gravity G and the contralateral leg chord φ_{Lk} is entered in each case.

1. A method for controlling an artificial knee joint comprising an upper part with an anterior face and a posterior face, a lower part which is mounted on the upper part so as to be pivotable about a knee axis and has an anterior face and a posterior face, a foot part arranged on the lower part, at least one sensor, a control device connected to the at least one sensor, and an actuator which is coupled to the control device and via which an achievable knee angle (KAmax) between the posterior face of the upper part and the posterior face of the lower part in the swim, phase can be set by the control device, characterized in that, on the basis of sensor data from the at least one sensor, it is concluded that a height difference (ΔH) of the foot part relative to a foot or a foot part of the contralateral side of a patient in their stance phase, or relative to the immediately preceding stance phase of the foot part during walking, is to be overcome, and the knee angle (KAmax) achievable in the swing phase is adjusted.

2. The method as claimed in claim 1, characterized in that, when a height difference (AH) increases counter to the direction of gravity (G), the achievable knee angle (KAmax) is reduced.

3. The method as claimed in claim 1, wherein the height difference (AH) is calculated or estimated from the trajectory of a trunk, a pelvis, hips and/or the knee axis of a leg aided by the artificial knee joint.

4. The method as claimed in claim 1. wherein the height difference (AH) is calculated or estimated over the vertical path of a hip joint of a leg aided by the artificial knee joint, over the vertical path of the knee axis (15) and/or the vertical path of the foot part (30).

5. The method as claimed in claim 1, wherein the height difference (ΔH) is determined via a hip angle (HA) of a leg aided by the artificial knee joint or the spatial orientation of the upper part and/or their time profiles.

6. The method as claimed in claim 1, wherein the height difference (ΔH) is determined via the time profile of the knee angle of a leg aided by the artificial knee joint.

7. The method as claimed in claim 1. wherein the height difference (ΔH) is calculated or estimated from the ratio of a horizontal movement of a trunk, a pelvis, the hip or the knee axis of a leg aided by the artificial knee joint to the hip angle (HA) or the spatial orientation of the upper part.

8. The method as claimed in claim 1, wherein the height difference (ΔH) is calculated from a determined knee angle (KAD) and a determined hip angle (HA).

9. The method as claimed in claim 1. wherein the achievable knee angle (KAmax) is set via an adjustable mechanical or hydraulic extension stop or a change in the movement resistance against knee extension.

10. The method as claimed in claim 1, wherein the spatial orientation of the lower part is used as a parameter for the achievable knee angle (KAmax).

11. The method as claimed in claim 1. wherein the height difference (ΔH) is determined or estimated from a knee angle (KAD) measured with a knee angle sensor on the artificial knee joint and/or a spatial position of the upper part and/or lower part measured via a spatial position sensor.

12. The method as claimed in claim 1, wherein the achievable knee angle (KAmax) is set in the swing phase and is maintained until a predetermined spatial position and/or movement of the lower part and/or upper part is reached, until an ankle joint angle (AA) and/or a force application point (PF) into the foot part is reached, and/or over a predetermined period of time.

13. The method as claimed in one of the preceding claims claim 1, wherein, after reaching a minimum hip angle (HA) and a movement reversal, the spatial orientation of the lower part is kept constant until detection of an initial contact, an axial force (FA) on the lower part and/or a change in an ankle joint angle (AA).

14. The method as claimed in claim 1, wherein walking up an incline, climbing stairs or otherwise negotiating a height difference during walking is detected via the time profile of the upper part orientation and/or the ratio of the upper part orientation to a translational horizontal movement of the knee axis, and the achievable knee angle (KAmax) is adjusted on the basis of the profile and/or the ratio.

15. The method as claimed in claim 1, wherein a flexion resistance in the swing phase, after reversal of the direction of movement of the lower part, is set to a level higher than when walking on level ground.

16. The method as claimed in claim 1, wherein, upon detection of walking up an incline, climbing stairs or otherwise negotiating a height difference (ΔH) during walking, the maximum achievable knee angle (KAmax) is reduced by 10° to 25°.

17. The method as claimed in claim 1. wherein the movement resistance against an extension movement of the knee joint is reduced continuously in the swing phase.

18. The method as claimed in claim 1, wherein the height difference (AH) is used as a parameter for the achievable knee angle (KAmax), and the actuator is activated or deactivated on the basis of this parameter.

19. A method for controlling an artificial knee joint, including the steps of:
providing a knee joint comprising an upper part with an anterior face and a posterior face, a lower part with an anterior face and a posterior face, the lower part being mounted on the upper part so as to be pivotable about a knee axis, a foot part arranged on the lower part, at least one sensor, a control device connected to the at least one sensor, and an actuator which is coupled to the control device and via which an achievable knee angle (KAmax) between the posterior face of the upper part and the posterior face of the lower part in the swing phase can be set by the control device, wherein:
obtaining sensor data from the at least one sensor;
determining from the sensor data that a height difference (AH) of the foot part relative to a foot or a foot part of the contralateral side of a patient in their stance phase, or relative to the immediately preceding stance phase of the foot part during walking, is to be overcome; and
adjusting the knee angle (KAmax) achievable in the swing phase, wherein when the height difference increases counter to the direction of gravity (G), the achievable knee angle (KAmax) is reduced.

20. A method for controlling an artificial knee joint, including the steps of:
providing a knee joint comprising an upper part with an anterior face and a posterior face, a lower part with an anterior face and a posterior face, the lower part being mounted on the upper part so as to be pivotable about a knee axis, a foot part arranged on the lower part, at least one sensor, a control device connected to the at least one sensor, and an actuator which is coupled to the control device and via which an achievable knee angle (KAmax) between the posterior face of the upper part and the posterior face of the lower part in the swing phase can be set by the control device, wherein:
obtaining sensor data from the at least one sensor;
determining from the sensor data that a height difference (ΔH) of the foot part relative to a foot or a foot part of the contralateral side of a patient in their stance phase, or relative to the immediately preceding stance phase of the foot part during walking, is to be overcome via the time profile of the knee angle of a leg aided by the artificial knee joint; and
adjusting the knee angle (KAmax) achievable in the swing phase, wherein when the height difference increases counter to the direction of gravity (G), the achievable knee angle (KAmax) is reduced.
